(19) United States
(12) Patent Application Publication
(10) Pub. No.: US 2011/0201869 A1
(43) Pub. Date: Aug. 18, 2011

(54) METHOD AND APPARATUS FOR ENHANCED TRANSDERMAL DIFFUSION

(75) Inventors: Jeffrey D. Edwards, Western Australia (AU); Maud Eijkenboom, Western Australia (AU)

(73) Assignee: OBJ LIMITED, Leederville (AU)

(21) Appl. No.: 12/736,771

(22) PCT Filed: May 7, 2008
(86) PCT No.: PCT/AU2008/000636

§ 371 Date: May 5, 2011

(51) Int. Cl. A61N 2/00

(52) U.S. Cl. 600/15; 600/9

(57) ABSTRACT

The present invention relates to a method and apparatus for enhanced transdermal diffusion of substances (such as pharmaceuticals, nutraceuticals, biopharmaceuticals and cosmeceutical) by application of anisotropic magnetic fields having distinctive, complex characteristics.

This application claims priority from Australian Provisional Patent Application No. 2007902418 filed 5 May 2007, the entire contents of which are incorporated herein by this reference.

### FIELD OF THE INVENTION

The present invention relates to a method and apparatus for enhanced transdermal delivery of substances (such as pharmaceuticals, nutraceuticals, biopharmaceuticals and cosmeceutical) by application of anisotropic magnetic fields having distinctive, complex characteristics.

### BACKGROUND OF THE INVENTION

The delivery of active agents in formulations (ie prepared as such as pharmaceuticals, nutraceuticals, biopharmaceuticals and cosmeceuticals) to treat and/or prevent disease, injury or disability is a cornerstone of modern human and animal medicine. Similarly the delivery of cosmetics to the skin is the cornerstone of the well-being and beauty market. For such substances to have a useful effect, they must be physically and/or chemically available to the target area at sufficient concentration to exert a beneficial effect.

A wide variety of different methods exist for delivery of active agent(s) to a patient. One such route is the transdermal route. This route brings about drug delivery across the skin barrier. Human skin is a complex, non-homogeneous membrane comprised of several skin tissue layers which extend inwardly from the outside skin surface and comprise stratum corneum, epidermis, dermis and sub-dermal tissue.

Transdermal delivery techniques are advantageous in that they enable local or systemic delivery of active agents. Such delivery seeks to provide a desired concentration of substance that is unaltered or unaffected by digestion or hepatic metabolism.

A number of techniques have been employed in order to facilitate diffusion of active agents through the skin. Chemical penetration enhancers can facilitate the diffusion of substances by modifying the diffusion coefficient of the stratum corneum using chemical means. Chemical penetration enhancers can be problematic due to unknown interaction with the active agent and its excipients and the potential for adverse side effects such as skin irritation. Moreover, penetration enhancers do not enhance diffusion by increasing molecular mobility, but only by affecting the dermal barrier.

Another diffusion enhancement technique is referred to as iontophoresis, in which an electrical energy gradient to accelerate the charged target active agent(s) across the skin. In this respect, skin possesses thermoelectrical properties which include both resistive and capacitive characteristics which cooperatively present impedance tending to oppose the desired current flow during therapy. These properties are understood to be dominated by the stratum corneum. The stratum corneum consists of multilayers of horny cells which possess relatively low water content and thus serves as a relatively good conductor. As such, a substantial percentage of the overall skin impedance is attributable to the stratum corneum.

Iontophoresis is only suitable to specific active agents with critical ionic structures. Additionally, iontophoresis requires the use of adhesive electrodes, which can become detached, leading to skin irritation and pain.

Other techniques to create mobility and/or direction in the movement of active agent(s) are possible, however they have been difficult to implement due to poor performance.

Magnetokinetics is one such technique. The use of repulsive magnetic forces has been employed in active agent delivery applications however its application has been limited to paramagnetic and paramagnetically coated substances.

Magnetophoresis is another technique that has been used in the transdermal delivery of substances such as benzoic acid; a specifically configured diamagnetic substance.

The present invention seeks to provide an improved delivery process for active agents that have a pharmaceutical, nutraceutical, biopharmaceutical, cosmeceutical or cosmetic benefit, which increases the directional penetration of these agents through the dermis.

### SUMMARY OF THE INVENTION

In accordance with an aspect of the present invention, there is provided a device adapted to deliver a active agent(s) to a subject, the said device comprising: an anisotropic array of magnetic material wherein the magnetic fields are directionally offset such that the polarity of each field is counter to that of the adjacent field and each pair of fields are separated, at least in part, by an insulator. Preferably, the magnetic materials are contained in a housing that does not interfere with the generated magnetic fields. In a broad form of the invention, the insulator does not travel the length of the magnetic materials, but it does provide a significant divide between each pair of fields.

In a more specific form of the invention, there is provided a device adapted to deliver a active agent(s) to a subject, the said device comprising: a biphasic array of magnetic materials wherein the magnetic fields generated by the magnetic materials are counter aligned such that the polarity of each field is counter that of the field adjacent to it and each pair of fields are separated by an insulator.

According to yet another more specific form of the invention, the inventive device will include a means for increasing kinetic energy in the active agent(s). Any means for inducing an increase in kinetic energy in the active agent(s) may be used in conjunction with the device. Preferentially, kinetic energy is increased using a thermal harnessing means that is able, to direct a subject's body heat to the active agent to be delivered to the subject, or to a formulation in which the active agent is included. Thus, the thermal harnessing means provides a way to direct a subject's body heat into the active agent, causing thermodynamic motion in the molecules making up the active agent. When the active agent(s) is thus activated the magnetic materials may then induce directional displacement in those molecules through the subsequent conversion of said thermodynamic motion in the molecules in the presence of a magnetic field.

The device of the invention provides a means for driving the passage of active agent(s) across the dermal barrier into a subject (including patient's) using the device. The utility of this device may be further enhanced by pairing it with an alternate drug delivery system that operates either in conjunction with, or in parallel with, the device, to promote the passage of active agents through the dermal barrier. Such alternate drug delivery systems may include, for example, iontophoresis, drug-adhesive matrix, micro-needles and sonophoresis.

In operation, the active agent(s) or a formulation including the active agent(s) is placed between the device and the subject (including a patient). Body heat is directed into the active agent either through contact of the active agent or a formulation which includes the active agent(s), with the skin and or through a thermal insulting means. Heat harnessed through this process will then drive thermodynamic motion in the active agent(s). The magnetic properties of the device of the invention can then induce directional displacement of the active agent(s) through the complex magnetic fields created by the present invention.

In a desirable form, the device is prepared as a wearable and thermally insulating occlusive dressing having anisotropic magnetic properties. The wearable and thermally insulating dressing may provide the thermal harnessing means for the purpose of effectively transferring body heat to any active agent placed against or in contact with the skin.

According to a particular form of the invention, the device is prepared from a flexible magnetic material of an insulating nature, which is placed over the active agent placed upon the skin. In such a form, the device may mould to the surface of the subject against which the device is placed.

In another a form of the invention, the device includes non-flexible magnetic material sewn or attached to insulating material that can be placed over substance molecules placed upon the skin. Preferably, said materials are shaped to fit the subject's extremity to which the device is to be applied.

In another a form of the invention, the active agent(s) are placed against or in closely proximity with the insulating and magnetic materials, on the side of the device that is in closest proximity to a subject's dermis. In this form, the active agent may be provided as a gel or in the form of a solid or semi-solid material that can be inserted between the device and the subject's dermis.

In another a form of the invention, the insulating and magnetic materials may be used as an alternative backing material to that commonly used in drug or cosmetic patch manufacture, thus enabling the present invention to be used in a standard drug-patch form.

Other aspects and advantages of the invention will become apparent to those skilled in the art from a review of the ensuing description, which proceeds with reference to the following illustrative drawings.

### BRIEF DESCRIPTION OF THE DRAWINGS

The present invention will now be described, by way of example only, with reference to the accompanying drawings.

FIG. 1 represents a graphical illustration of a form of the device of the invention as applied to an individual.

FIG. 2 represents an alternate illustration of a form of the device of the invention as applied to an individual.

FIG. 3 represents a cross sectional view of a device as applied to an individual, which illustrates the deployment process for the enhanced delivery patch.

FIG. 4 illustratively depicts the results of experiments carried out employing the process of the invention, which shows enhanced in vitro transdermal diffusion of Tetracaine in a Franz Cell diffusion study. The data show enhanced diffusion of 2 (FIG. 4A) and 4% Tetracaine (FIG. 4B) through excised epidermis under the influence of the present invention when compared to passive diffusion.

FIG. 5 illustratively depicts the results of experiments carried out employing the process of the invention, which shows enhanced in vitro transdermal diffusion of Sumatriptan in a Franz Cell diffusion study.

FIG. 6 presents a depiction of a specific representation of an embodiment of the invention.

FIG. 7 illustratively depicts the results of experiments carried out to compare the present invention versus passive diffusion.

FIG. 8 illustratively considers Methyl Nicotinate delivery wherein: one treatment incorporated a 4 cm² piece of the present invention having a field strength of 480 gauss and a pole density of 300 poles per meter on top of the Methyl Nicotinate application, whereas the second treatment consisted out of a 120 gauss field with a 500 poles per meter pole density on top of the Methyl Nicotinate.

### DESCRIPTION OF THE INVENTION

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. size, concentration etc). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### Preferred Embodiments

The inventors of the present invention have revealed that normal thermodynamic forces induce random movement and orientation in therapeutic active agent(s). When such active agent(s) have negative magnetic susceptibility, they may be influenced by a magnetic field resulting in repulsive forces that may cause the molecules forming the active agent(s) to be subject to collective diffusion rather than simple diffusion or brownian motion. That is, an active agent(s) diamagnetic susceptibility, being a measure of the amount of repulsive force created by a magnetic field acting upon an active agent(s) electron orbit(s), will cause a repulsive force to be generated in a direction away from the applied magnetic field. This creates directional movement of active agent(s) molecules within a magnetic field.

The inventors of the present invention have also revealed that when molecules in motion are exposed to an array of alternating magnetic polarity, current of opposite polarity is generated in differing regions, due to the magnetic vector change. Such polarity differences create differential current flows and when this occurs within a magnetic field, a further force is created that enhances the otherwise weak diamagnetic force to create a total repulsive force of sufficient magnitude to influence the movement of molecules during diffusion and partitioning.

In accordance with an aspect of the present invention there is provided a device adapted to deliver an active agent(s) to a subject comprising: an anisotropic array of magnetic materials wherein the magnetic fields are directionally offset such that the polarity of each field is counter that of the adjacent field and each pair of fields are separated, at least in part, by an insulator.

According to the invention, magnetic materials include, without limitation:
a. Arrangements where individual segments or sections of magnetized ferromagnetic materials are assembled in the configuration described herein; and
b. Arrangements where magnetic particles or elements are disposed in a solid or semi-solid matrix or base and the required magnetic patterns as impressed upon the ferromagnetic particles.

The present invention may be constructed using a range of magnetic materials exhibiting ferromagnetic properties. Such materials may include Iron, Cobalt or Nickel with a metalloid component such as Boron, Carbon, Silicon, Phosphorus or Aluminum. Alternately rare-earth materials such as Neodymium or Samarium-cobalt may also be used. Such ferromagnetic materials may be deployed as rigid elements within a device or encapsulated in a flexible matrix such as rubber or silicone.

Preferably, the magnetic materials are contained in a housing that does not interfere with the generated magnetic fields.

In the broadest form of the invention, the insulator is not required to travel the length of the magnetic materials; however it is preferable that the insulator provides a significant divide between paired magnetic fields.

In addition to separating adjacent paired magnetic materials the insulator may restrict the escape of thermal energy from the skin to the outer environment, and or provide a means to allow even distribution of thermokinetic energy to the active agent. The insulator may be: (a) a separate element(s) inserted between individual segments or sections; or (b) a property of the substrate; or (c) a property of the ferromagnetic particles.

In accordance with a more specific aspect of the invention there is presented a device adapted to deliver a active agent(s) to a subject comprising: a biphasic array of magnetic materials wherein the magnetic fields generated by the magnetic materials are counter aligned such that the polarity of each field is counter that of the field adjacent to it and each pair of fields are separated by an insulator.

In a more specific form of the invention, the device will include a polymer coating comprising a backing layer that is substantially impermeable to active substances located adjacent the magnetic material and a matrix layer also adjacent the magnetic material containing active agent(s) or a plurality of different active agent(s) that are thermoactivatable and are to be delivered to a subject.

The matrix is preferentially prepared from a polymer or copolymer prepared from e.g., polyisobutylene, ester of polyvinyl alcohol, polyacrylic and polymethacrylic acid esters, natural rubber, polymers of styrene, isoprene, and styrene-butadiene or silicone polymers, resin components, such as, saturated and unsaturated hydrocarbon resins, derivatives of abietyl alcohol and of beta-pinene, plasticizers, such as phthalic acid esters, triglycerides and fatty acids, as well as a series of other substances known to those skilled in the art.

Matrix biocompatible polymers that might be used in the invention include compounds such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAID), and other polymers such as those disclosed in U.S. Pat. Nos. 6,667,371; 6,613,355; 6,596,296; 6,413,536; 5,968,543; 4,079,038; 4,093,709; 4,131,648; 4,138,341; 4,180,646; 4,304,767; 4,946,931, each of which is expressly incorporated by reference herein in its entirety.

The matrix may also be prepared from thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The matrix may also be a hydrogel, being a gel prepared with hydrophilic polymers, and these materials are well known in the art, frequently being used as part of biomedical electrodes, such as are described in U.S. Pat. Nos. 6,631,294 and 6,845,272, the contents of which are incorporated herein by reference. Examples of hydrophilic polymers useful for the preparation of hydrogels are polyacrylate, polymethacrylate, polyacrylamide, poly(vinyl alcohol), poly(ethylene oxide), poly(ethylene imine), carboxy-methylcellulose, methylcellulose, polyacrylamide sulphonic acid), polyacrylonitrile, poly(vinyl-pyrrolidone), agar, dextran, dextrin, carrageenan, xanthan, and guar. The preferred hydrogels are acrylates and may be, for example, preferably made from acrylic esters of quaternary chlorides and/or sulfates or acrylic amides of quaternary chlorides; polymers of this type are disclosed in U.S. Pat. No. 5,800,685, incorporated herein by reference. The hydrophilic polymers will generally constitute from about 1 to about 70%, preferably about 5 to about 60%, more preferably about 10 to about 50%, by weight of the hydrogel.

The active agent(s) delivered by the device of the invention may cover the entire region of the contact zone between the device and the skin or alternatively may be formed in islands therein. In a preferred form, the active agent(s) are located between the inventive device and a subject's dermis.

The process of enhanced delivery by the present invention involves the utilization of magnetic principles to apply forte upon active agent(s) in such a manner as to ensure that the force acting upon the agents is different from that acting upon the molecules of the vehicle, gel or solvent. As a result, one method of improving the utility of the invention is to select or chemically alter the magnetic sensitivity of the active agent or that of the vehicle, gel or solvent in which it is located with the view to enhancing the differences in magnetic sensitivity between the two entities. By way of example, the additional of a light ester such as phenxyethyl acrylate to a diethylaminoethyl acrylate polymer may act to increase the magnetic susceptibility of the polymer and by doing so increase the delivery of a diamagnetic target molecule from that vehicle, gel or solvent.

Suitable active agent(s) that can be delivered by the invention include any active agent(s) exhibiting negative magnetic susceptibility and any active agent(s) having therapeutic, cosmetic, restorative or beneficial properties when administered transdermally, perdermally and interdermally

Classes of active agents, include, for example, proteins, peptides, nucleotides, anti-obesity drugs, corticosteroids, analgesics, anti-fungal agents, oncology therapies, cardiovascular agents, anti-inflammatory agents, non-steroidal anti-inflammatory agents, anti-arrhythmic agents, antibiotics, anticoagulants, antidepressants, antidiabetic agents, antiepileptics, antihistamines, anti-hypertension agents, anti-neoplastic agents, immunosuppressants, anti-thyroid agents, antiviral agents, sedatives, astringents, beta-adrenoceptor blocking agents, diuretics, muscle reactants, prostaglandins, sex hormones, anti-allergic agents, stimulants, vasodilators, xanthenes, antioxidants, vitamins, nutrients, skin restorative agents and those active agents delivered as nutraceuticals, cosmeceutical or cosmetics to or through a dermal surface.

The following provides an illustrative list of some of the active agents that may be used such as with the apparatus of the present invention:
a. parasympathicolytics scopolamine, and benactyzine and the like;
b. cholinergics such as physostigmine, nicotine and the like;
c. monoamine oxidase inhibitors such as tranylcypiomine, selegiline and the like;
d. sympathomimetics such as ephedrine, D-norpseudoephedrine, salbutamol, fenfluramine and the like;
e. adrenergic blockers and anticympathotonics such as propanolol, timolol, bupranolol, clonidine, dihydroergotamine, naphazoline and the like;
f. H₁ Receptor Antagonists such as: terfenadine and the like;
g. H₂ Receptor Antagonists such as: Famotidine, Cimetidine and Ranitidine Hydrochloride and the like;
h. antispasmodics such as Hyoscine Butylbromide, Hyoscine Hydrobromide, atropine, methscopolamine bromide and the like;
i. beta-adrenergic agents such as: Antagonists—Esmolol hydrochloride, Propranolol HCl and Atenolol. (β-adrenergic agonist); agonists DL isoproterenol hydrochloride and the like;
j. diuretic effects agents such as Frusemide and the like;
k. anti-arrhythmic agents such as; Amiodarone hydrochloride, Verapamil hydrochloride, Procailnamide hydrochloride, Disopyramide, Flecamide acetate, and Lignocaine hydrochloride and the like;
l. adrenergic stimulants such as: Adrenalin, Metaraminol bitartrate, Dobutamine hydrochloride, Isoprenaline hydrochloride, Noradrenaline acid tartrate and Dopamine hydrochloride and the like;
m. antimigraine preparations such as: Dihydroergotamine mesylate, mazindol, phentermine and Sumatriptan succinate and the like;
n. other cardiovascular agents such as: Indomethacin Methyldopate HCl, Hydralazine hydrochloride, isosorbide dinitrate, Clonidine hydrochloride, Verapamil, Glyceril Trinitrate, Rauwolfia alkaloids, organic nitrates, pentaerythritotetranitrate, Diazoxide and Sodium nitroprusside and the like;
o. antihistamines (e.g., diphenhydramine, clemastine; terfenadine)
p. sedatives and hypnotics such as: chlorpromazine HCl, clozapine, mesoridazine, metiapine, reserpine, thioridazine Midazolam, Paraldehyde, propofol, codeine, phenobarbital, sodium pentobarbital, sodium secobarbital, chlordiazepoxide, diazepam, meprobamate and temazepam and the like;
q. anti-anxiety agents such as: Diazepam, properidol, dopamine antagonist such as Haloperidol decanoate, and the like;
r. anti-depressants such as desipramine hydrochloride and the like;
s. psychotropic agents such as 5-hydroxytryptamine receptor antagonists (e.g. ondanstetron, sumatriptan, naratryptan), ergotamine tartrate plus caffeine, or methylsegide and the like;
t. movement disorder agents such as Benztropine mesylate, Phenyloin sodium, Phenobarbitone sodium and Clonazepam and the like;
u. anti-Parkinson agents such as L-DOPA and the like;
v. narcotic analgesics such as Fentanyl citrate, Sufentanyl, Alphentanyl, Morphine Sulphate, dihydrocodienone, hydromorphine, meperidine, Pethidine hydrochloride, Phenoperidine hydrochloride, Papaveretum, Methadone hydrochloride and Buprenophine hydrochloride and the like;
w. narcotic antagonists such as naltrexone and naloxone and the like;
x. non-steroidal agents such as salicylates, acetaminophen, d-propoxyphene, Indomethacin, Naproxen and Ketorolac trometamol and the like;
y. other Analgesics such as (cyclooxygenase inhibitors), fenamic acids, Pircadcam, COX-2 inhibitors, diclofenac), rofecoxib, and Ibuprofren and the like;
z. hormonal preparations such Menopausal Gonadotrophin, Growth Hormone—Somatropin, Desmopressin acetate, Bromocriptine mesylate, Octreotide, Insulin, Glibenclamide, Metformin hydrochloride, Glipizide and Tolbutamide and the like;
aa. agents acting on the uterus such as: Oxytocin and the like;
bb. steroids such as sulfonamides, triamcinolone, betamethasone, budesonide, cortisone, dexamethasone, hydrocortisone, methylprednisolone, prednisolone, prednisone sodium phosphate, fluorometholone, rimexolone, medrysone alcohol, 11-desoxcortisol, and anecortave acetate and the like
cc. testosterone and testosterone propionate, progesterone and estrogens such as diethyl stilbestrol, 17-beta-estradiol, estrone, ethinyl estradiol, mestranol, and the like;
dd. prostaglandins such as Ritodrine hydrochloride and Salbutamol sulfate and the like;
ee, antitussives such as dextromethorphan, noscapine and the like;
ff. anti-inflammatory agents such as hormonal agents, clobetasol, dexamethasone, acetyl salicylic acid, glycyrrhizic acid or glycyrrhetic acid, hydrocortisone, prednisolone, prednisone, non-hormonal agents, allopurinol, aspirin, indomethacin, phenylbutazone and the like;
gg. bronchospasm relaxants such as Aminophylline, Theophylline, Salbutamol sulfate, Orciprenaline sulfate, Ipratropium bromide, Fenoterol hydrobromide, Terbutaline sulfate and Adrenaline acid tartrate and the like
hh. antimalarials such as 4-aminoquinolines, 8-aminoquinolines, pyrimethamine and the like;
ii. antibiotics such as the cephalosporins, chloranphenical, gentamicin, Kanamycin A, Kanamycin B, the penicillins, ampicillin, streptomycin A, antimycin A, chloropamtheniol, metromidazole, oxytetracycline penicillin G, the tetacyclines and the like;
jj. sexual dysfunction agents such as sildenafil citrate, tadalafil and vardenafil and the like;
kk. cytotoxic drugs such as thiotepa, chlorambucil, cyclophosphamide, melphalan, methotrexate and the like; Cardio-ionatropic agents such as Digoxin and the like;
ll. vitamins and nutrients including essential amino acids and the like;
mm. electrolyte replacements such as potassium chloride and the like;
nn. peptides with either therapeutic benefit or cosmetic benefit comprising between two and 20 amino acid residues, preferably, between three and 10 amino acid residues (cosmetic peptides such as palmitoyl pentapeptide or argireline and the like will—preferably they have beneficial effect on skin cells eg whitening, free-radical scavenging, anti-aging, stimulation of collagen synthesis, moisturizing, antimicrobial, anti-inflammatory, or anti-irritant and the like;
oo. astringents, such as clove oil, menthol, camphor, eucalyptus oil, eugenol, menthyl lactate, witch hazel distillate and the like;
pp. antioxidants or free-radical scavengers, such as ascorbic acid, its fatty esters and phosphates, tocopherol and its derivatives, N-acetyl cysteine, sorbic acid and lipoic acid and the like;
qq. anti-acne agents, such as salicylic acid, and benzoyl peroxide;
rr. antimicrobial or antifungal agents such as caprylyl glycol, triclosan, phenoxyethanol, erythromycin, tolnaftate, nystatin or clortrimazole;
ss. chelating agents, such as EDTA and the like;
tt. topical analgesics, such as benzocaine, tetracaine, lidocaine or procaine and the like;
uu. central analgesics such as fentanyl, sufentanil and the like;
vv. antirheumatics such as indomethacin, piroxicam, lornoxicam
ww. anti-aging/anti-wrinkle agents, such as retinoids or hydroxy adds;
xx. skin lightening agents, such as licorice, ascorbyl phosphates, hydroquinone or kojic acid and the like;
yy. skin-conditioning agents such as humectants, including miscellaneous and occlusive and the like;
zz. antiirritants, such as cola, bisabolol, aloe vera or panthenol and the like;
aaa. anti-cellulite agents, such as caffeine and other xanthines and the like;
bbb. humectants, such as alkylene polyols or hyaluronic acid and the like;
ccc. emollients, such as oily esters or petrolatum and the like;
ddd. sun protecting agents (organic or inorganic), such as avobenzone, oxybenzone, octylmethoxycinnamate, titanium dioxide or zinc oxide;
eee. exfoliating agents (chemical or physical), such as N-acetyl glucosamine, mannose phosphate, hydroxy adds, lactobionic acid, peach kernels, or sea salts and the like; and
fff. self-tanning agents, such as dihydroxyacetone.

The above list of active agent(s) may be applied in a controlled manner, using the present invention. This list is not exhaustive. Preferably, any active agent(s) that can be delivered systemically or topically can potentially be delivered using the present invention.

While the active agent(s) may be provided and used alone with the device, in many situations the active agent will be included in a formulation either alone or in combination with one or more active agents. Where the formulation is to provide a pharmaceutical and or biopharmaceutical benefit, the number of active agent(s) included in the formulation may preferentially be quite selective. Where the formulation provides a nutraceuticals, cosmetic and/or cosmeceutical, the number of active agents may be much greater in number.

The formulation employed in the delivery process may include additives such as other buffers, diluents, carriers, adjuvants or excipients. Any pharmacologically acceptable buffer that is magnetically inert or neutral or which has a magnetic susceptibility that is either paramagnetic in nature or greater than that of the active agent(s) being delivered, may be used, e.g., tris or phosphate buffers. Other agents may be employed in the formulation for a variety of purposes. For example, buffering agents, preservatives, co-solvents, surfactants, oils, humectants, emollients, chelating agents, stabilizers or antioxidants may be employed. Water soluble preservatives which may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80. Other vehicles that may be used include, but are not limited to, polyvinyl alcohol povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc.

The indications, effective doses, contraindications, vendors etc, of the active agents in the formulations are available or are known to one skilled in the art.

The active agents may be present in individual amounts of from about 0.001 to about 5% by weight and preferably about 0.01% to about 2% by weight. Suitable water soluble buffering agents that may be employed are sodium carbonate, sodium borate, sodium phosphate, sodium acetate, sodium bicarbonate, etc., as approved by the US FDA for the desired route of administration. These agents may be present in amounts sufficient to maintain a pH of the system of between about 2 to about 9, preferably about 4 to about 8, more preferably 4.5, 5, 5.5, 6, 6.5, 7 or 7.5 (or any pH in between). As such the buffering agent may be as much as about 5% on a weight to weight basis of the total formulation. Electrolytes such as, but not limited to, sodium chloride and potassium chloride may also be included in the formulation where appropriate.

In a highly preferred form of the invention, a topical formulation for delivery to a subject is prepared by selecting a desired amount of active agent. The agent is then preferably placed in a suitable delivery matrix. The amount of the active agent to be administered and the concentration of the compound in the topical formulation depends upon the diluent, delivery system or device selected, the clinical or cosmetic condition of the subject, the side effects and the stability of the active agent in the matrix.

In the process of delivery of the active agent, the kinetic energy of the active agent(s) is first increased. Any means for inducing an increase in the kinetic energy in the active agent may be used in conjunction with the device described herein. Preferentially, this is achieved using body heat. Such heat can raise the temperature of therapeutic molecules when placed on the skin from around 20° C. to close to 37° C.

According to a preferred form of the invention, the means for increasing kinetic energy in the active agent will preferentially be a thermal harnessing means that is adapted to transfer body heat to any active agent(s) placed against or in contact with a subject's dermis. Thus, the thermal harnessing means provides a way to direct a subject's body heat into the active agent(s) causing thermodynamic motion in substance molecules. When such a state is induced the magnetic materials can then induce directional displacement in substance molecules through the subsequent conversion of said thermodynamic motion in substance molecules in the presence of a magnetic field.

The thermal harnessing means may be part of the identified insulator or a specific property of the magnetic materials employed or may be a separate component of the invention. In a preferred form, the thermal harnessing means will be part of the insulator. In an alternate preferred form, the thermal harnessing means is a distinct component associated with the device which preserves and provides a means to harness the thermal energy generated by the body. When provided in this alternate form the thermal harnessing means may be a bandage or the like which restricts dissipation of the body heat from the region surrounding where the device is positioned on a subject.

The resultant increase in kinetic energy in the active agent causes increased rotational movement of paired electron orbits in the molecules of the active agent. When this increase in rotational kinetic energy is constrained in a magnetic field repulsive forces are generated which act to displace the molecules in the active agent away from the magnetic field source, towards the skin. In addition, the change in electron shell orbits results in induced surface charge, turning the molecules into temporary charge carriers. The motion of the charged carrier within a magnetic field as provided by the present invention will further enhance the repulsive force and molecular displacement. Furthermore, the thermal gradient created by the entrapment of thermal energy causes the charge carrier molecules to migrate along the thermal gradient that exists between skin temperatures and the cooler outer surface, in accordance with the Nernst-Ettingshaussen effect. Such thermal driven movement of charge carriers within a magnetic field will generate a further perpendicular force to enhance that movement. These forces while being individually weak forces, contribute to the total displacement forces acting upon drug molecules. Therefore, by exposing molecules to a magnetic field and a temperature gradient normal to each other, organized alignment in the molecules is induced in the molecules with the kinetic energy component of the molecular moment being converted into diamagnetic repulsion in a direction away from the magnetic field source.

According to the invention the generated weak repulsive forces can be further enhanced by employing counter aligned magnetic fields, which facilitate the generation of current flow or streaming potentials in the active agent.

Rotating electron orbits of dissolved molecules rotating in a magnetic field, act to generate small but significant electrical fields of polarity specific to the orientation of the magnetic field. An applied temperature difference causes charged electrons in the therapeutic material, to travel from one magnetic polarity, which effectively charges the molecules. Charged molecules (for example conduction-band electrons) will move along temperature gradients due to static and the relationship between temperature and kinetic energy. When there is a magnetic field transverse to the temperature gradient and the carriers are electrically charged, they experience a force perpendicular to their direction of motion (also the direction of the temperature gradient) and to the magnetic field. This process effectively increases the beneficial repulsive force subjected on the molecules.

To generate the necessary electrical current, the magnetic field is provided in the form of a series of closely aligned alternatively oriented fields, as disclosed. By having alternative polarity magnetic fields in close proximity to each other, adjacent regions create electrical fields of opposite polarity resulting in current flow or streaming potentials in the therapeutic molecules. The current flow or streaming potentials mobilize and direct the molecules through the dermal barrier.

The inventors of the present invention have discovered that such apparatus and methods results in enhanced transdermal diffusion of substance molecules.

In a highly specific form of the invention the device consists of a magnetic material prepared as a barium ferrite bi-phasic polymer, comprising parallel bands of magnetic material, separated by an insulating material, aligned in such a manner that each band is of opposite polarity. Desirably, the magnetic material the barium ferrite bands are constrained in a flexible polymer material of between 0.5 and 1.00 mm height, between 2.00 and 3.00 mm width and oriented in continuous strips having an insulator of 0.1 mm thickness separating each component. In this respect the top and bottom “films” are provided only to hold the materials in place.

For enhanced transdermal diffusion of active agents the agent is exposed to magnetic fields between 5 mT to 100 mT. Preferably, the magnetic field attributes are selected according to the specific electron bond characteristics present in the therapeutic molecules to be delivered to the patient. More particularly, the magnetic field attributes are preferably controlled to deliver periodic delivery of energy which more effectively interacts with the thermomagnetic characteristics of the active agent to overcome the impedance of the skin tissue being treated.

The device of the invention provides a means for driving the passage of active agents across the dermal barrier into a subject. The utility of this device may, however, be further enhanced by pairing it with techniques or processes designed to overcome the normal dermal barrier effect of the stratum corneum by alternate means. Such technique or process may include for example, iontophoresis, drug-adhesive matrix, chemical enhancement, micro-needles and sonophoresis. In such cases, said techniques or processes when used either in conjunction with or parallel with the device would promote the passage of active agents through the dermal barrier.

In operation, an active agent is placed between the device and the subject therein providing a means for enhanced transdermal diffusion of at least a substance into a subject.

In a desirable form, the device of the invention is provided as a wearable and thermally insulating occlusive dressing having magnetic properties. The wearable and thermally insulating dressing being provided for the purpose of effectively transferring body heat to any active agent(s) placed against or in contact with the skin. The body heat being converted into thermodynamic motion in substance molecules. The magnetic properties of the apparatus being provided for the purpose of inducing directional displacement in substance molecules through the subsequent conversion of said thermodynamic motion in substance molecules in the presence of a magnetic field.

According to a particular form, the invention employs flexible magnetic material of an insulating nature to be placed over substance molecules placed upon the skin.

In another arrangement, the present invention employs non-flexible magnetic material sewn or attached to insulating material that can be placed over substance molecules placed upon the skin.

In yet another arrangement, substance molecules may be placed or closely associated with the insulating and magnetic materials to form a patch-like device that can be applied as device to the area requiring treatment.

In the present invention, the application of specifically selected magnetic fields to specifically selected substance molecules results in directions movement of substance molecules independent of carrier, solvent or base molecules.

### Non-Limiting Illustration of the Invention

Further features of the present invention are more fully described in the following non-limiting Examples. This description is included solely for the purposes of exemplifying the present invention. It should not be understood as a restriction on the broad description of the invention as set out above.

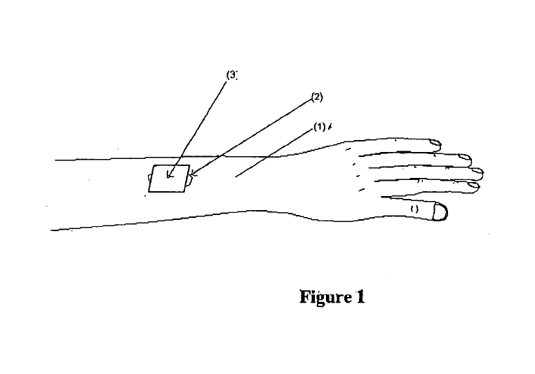

FIG. 1 represents a graphical interpretation of one possible embodiment of the present invention in which Ointment containing a active agent (2) is place on a subjects arm (1). A dressing (3) having magnetic and insulating properties is applied over the ointment to provide enhanced transdermal delivery of the active agent.

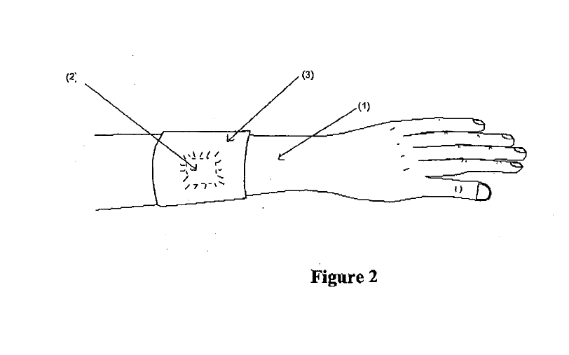

FIG. 2 represents another embodiment in which the magnetic source (2) is integrated into thermally insulating materials such as neoprene and deployed as a wearable orthotic (3) for placement on the arm (1) or such other body part as may require the enhanced transdermal delivery of substance molecules.

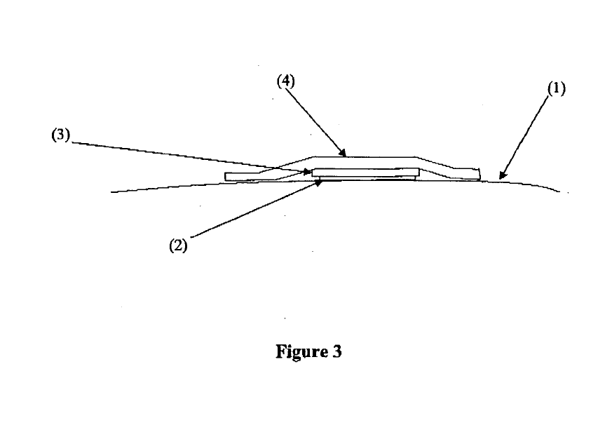

FIG. 3 shown a further embodiment in which ointment (2) may be impregnated, bound or adhered to the magnetic source (3) or to the orthotic material (4) and deployed as an enhanced drug delivery patch to the arm (1) or such other body part as may require the enhanced transdermal delivery of substance molecules.

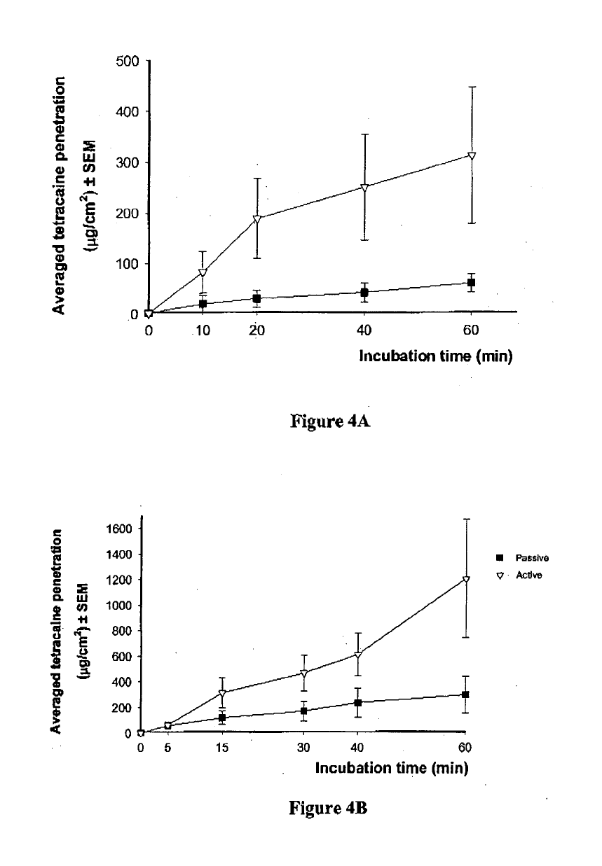

FIG. 4 is a plot of the substance Tetracaine through excised piglet ear epidermis in an in vitro Franz Cell diffusion study. Epidermal membranes were obtained by heat separation (60 sec at 60° C., Kligman and Christophers 1963). Skin integrity was determined by conductance measurement before the experiment. Skin sections from each ear were matched over the groups per experiment. Epidermal membranes were placed between the donor and receptor compartments of Franz-type diffusion cells with the stratum corneum facing the donor chamber. The epidermal membranes were equilibrated with the receptor solution (USP PBS, pH 7.4) for 30 min. The receptor compartment of the cell was immersed in a water bath at 37±0.5° C., and stirred every 5 min throughout the experiment. After equilibration, the donor solution consisting of 1 mL of 2 (FIG. 4a) or 4% Tetracaine HCL (FIG. 4B; Sigma) in PBS was added, and the present invention was suspended 3 mm above the surface of the donor fluid (i.e. 8 mm above the epidermal surface). The passive diffusion cells received the same donor drug concentration and were covered with parafilm only, i.e. no material of the present invention was applied. The administration period was 0-1 hr. Samples of the receptor solution were removed and replaced with fresh buffer over a 60 minute period. All samples were analysed for Tetracaine hydrochloride content by spectrophotometric detection (absorbance at 229 nM). The cumulative amount of Tetracaine hydrochloride in the receptor compartment versus time was plotted for both experiments. After the diffusion experiment the skin, still fixed in the diffusion chamber, was stained with standard HE dye. The diffusion area was investigated under a microscope for the occurrence of damage to the skin. Data derived from epidermis with low skin integrity was excluded from analysis and graphical depiction.

Results: The graphs show enhanced diffusion of 2 (FIG. 4A) and 4% Tetracaine (FIG. 4B) through excised epidermis under the influence of the present invention when compared to passive diffusion.

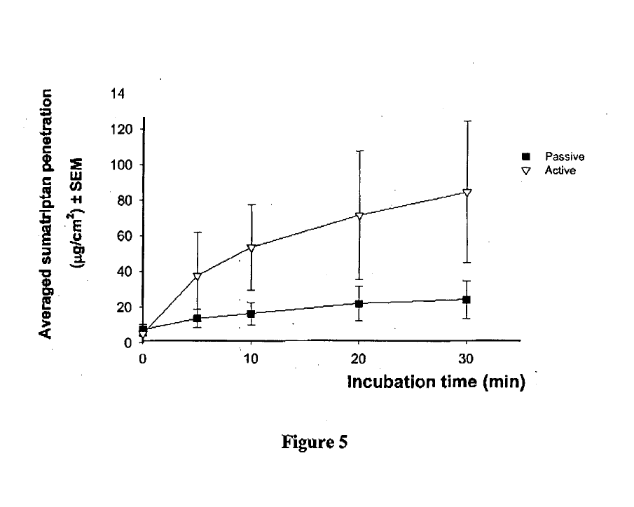

FIG. 5 is a plot of the substance Sumatriptan through excised piglet ear epidermis in an in vitro Franz Cell diffusion study. Sumatriptan succinate at a concentration of 2 mg/ml was applied to the donor compartment of diffusion cells, with PBS in the receptor compartment (3.0 mL; stirred continuously; water bath at 37° C.). The experimental procedures were as reported for FIG. 4, with the exception of the treatments that were compared, the drug applied, and the duration of the experiment (30 min). The present invention, constructed to 1.5 mm (thickness)×10 mm (diameter) dimensions, was applied from time 0-30 min. Samples of the receptor solution were removed and replaced with fresh buffer over a 30 minute period. All samples were analysed for Sumatriptan content by spectrophotometric detection (absorbance at 285 nM). The cumulative amount of Sumatriptan in the receptor compartment versus time was plotted. The graph shows enhanced diffusion of Sumatriptan through excised epidermis under the influence of the present invention when compared to passive diffusion.

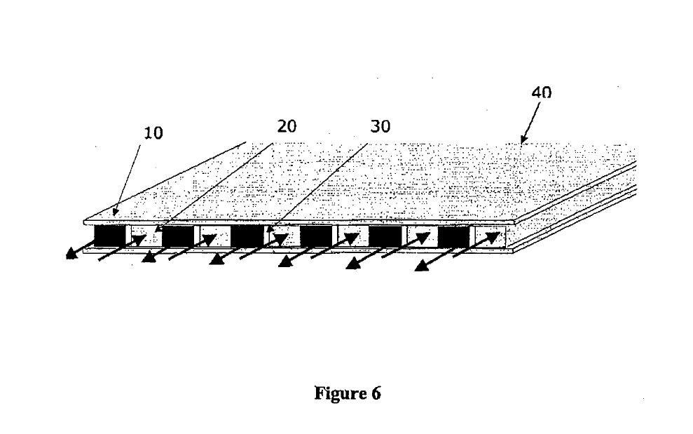

FIG. 6 depicts the counter aligned magnetic materials such that an opposite polarity resides adjacent each material. Located between each pair of magnetic material (10, 20) is an insulator (30) and both magnetic materials and the insulator are housed in a polymer matrix (40), which is located above and below the magnetic material. As depicted in FIG. 6, the magnetic materials are provided in rod like form and are co-aligned in a north-south (10) and south-north (20) configuration with each pair of magnets being separated by an insulator (30). It will be appreciated that the invention is not so limited and other configurations of the magnetic material may be employed in the invention.

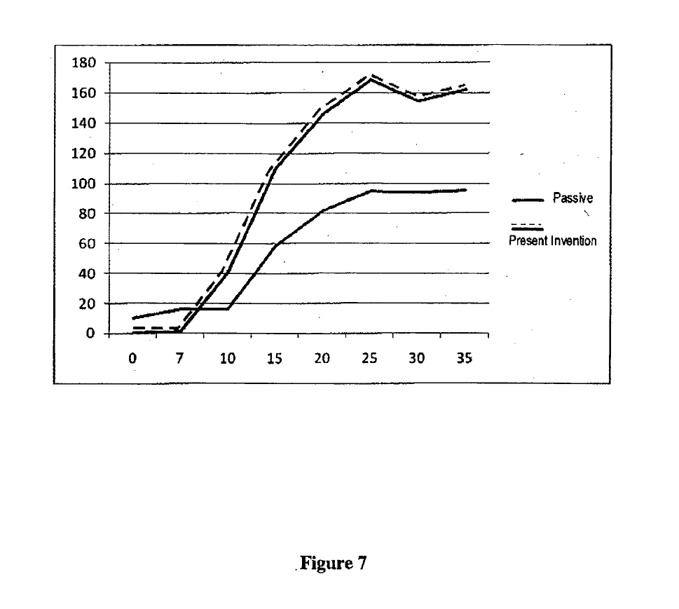

FIG. 7 depicts the enhancing effects of the present invention on the delivery of Methyl Nicotinate through human skin in vivo. The Methyl Nicotinate Laser-Doppler vasodilation model was used (see Hollaway et al, J. Invest Dermatol 69:306-309, 1977; Wester et al., J. Invest Dermatol 83:515-517, 1984). This model has often been used to demonstrate the transdermal delivery efficiency of other transdermal drug delivery processes such as sonophoresis and chemical permeation enhancement. In short, successful transdermal delivery of the rubifacient Methyl Nicotinate through the skin results in pronounced vasodilation, increased perfusion and erythema. The change in perfusion has been shown to be proportional to the bioavailability of the transdermally delivered Methyl Nicotinate and is therefore a measure of transdermal penetration (Guy et al Pharm. Res. 1:76-81, 1984). The Methyl Nicotinate induced vasodilation can be measured using a non-invasive Laser Doppler Flow or Perfusion meter with data being provided as a numeric value equitable to local in vivo blood perfusion.

The present experiment involved the saturation of 4 cm² tissue paper units with Methyl Nicotinate (5 mmolar in PBS). The Methyl Nicotinate tissues were applied to the skin of the volar forearm. The present invention (4 cm²) and the passive surrogate units were placed over the Methyl Nicotinate soaked paper units. For 5 minutes one active and one passive unit were applied adjacently onto the skin of the forearm. Subsequently, all materials were removed from the skin and the skin was patted dry. Laser Doppler perfusion probes (Moor Instruments Laser Doppler Perfusion Meter) were placed over both areas and blood perfusion rates were monitored for a 30 minute period.

Results: Data in FIG. 7 demonstrates that the present invention is effective at enhanced delivery of Methyl Nicotinate through human skin in vivo when compared with passive diffusion.

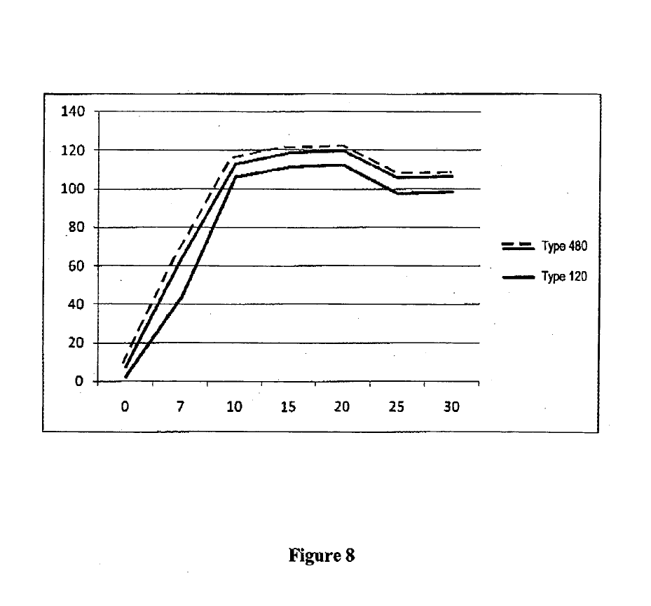

FIG. 8 depicts that an increase in pole density of the present invention can compensate for lower magnetic flux, and achieve similar transdermal drug delivery levels as a stronger magnetic flux field with less pole density.

It is generally believed that only materials with high magnetic flux are suitable for diamagnetic drug delivery. The inventors of the present invention have discovered that a higher density or number of poles per unit of area can deliver target molecules through the skin while using a low magnetic flux, without overall loss of performance.

The study depicted in FIG. 8 used the same Methyl Nicotinate protocol as the study depicted in FIG. 7 other than for a lower molar concentration of 2 milli-molar. Two groups were compared: one treatment incorporated a 4 cm² piece of the present invention having a field strength of 480 gauss and a pole density of 300 poles per meter on top of the Methyl Nicotinate application, whereas the second treatment consisted out of a 120 gauss field with a 500 poles per meter pole density on top of the Methyl Nicotinate.

Results: FIG. 8 illustrates that both applications produced a similar Methyl Nicotinate diffusion, even though the magnetic flux of the first treatment exceeded the second treatment by 4-fold. This data supports the investors' discovery that a relationship exists between peak magnetic flux and pole density such that a reduction in one may be compensated by a corresponding increase in the other.

Numerous variations and modifications will suggest themselves to persons skilled in the relevant technical arts, in addition to those already described, without departing from the basic inventive concepts. All such variations and modifications are to be considered within the scope of the present invention, the nature of which is to be determined from the foregoing description.

1. A device adapted to deliver an active agent(s) or formulation including active agent(s) to a subject, said device comprising:
(a) an anisotropic array of magnetic material wherein the magnetic fields generated by the magnetic materials are directionally offset such that the polarity of each field is counter to that of the adjacent field and each pair of fields are separated, at least in part, by an insulator; and
(b) a thermal harnessing means for increasing kinetic energy in the active agent(s) or formulation including active agent(s).

2. A device adapted to deliver an active agent(s) or formulation including active agent(s) to a subject, said device comprising:
(a) a biphasic array of magnetic materials wherein the magnetic fields generated by the magnetic materials are counter aligned such that the polarity of each field is counter that of the field adjacent to it and each pair of fields are separated by an insulator; and
(b) a thermal harnessing means for increasing kinetic energy in the active agent(s) or formulation including active agent(s).

3. The device according to claim 1 wherein the device is secured to the subject by a wearable and thermally insulating occlusive dressing or patch.

4. A method of delivering an active agent(s) or a formulation including the active agent(s) to a patient comprising the step of:
(a) locating said active agent(s) or formulation including active agent(s) between a device and the subject, wherein the device comprises an anisotropic array of magnetic material wherein the magnetic fields generated by the magnetic materials are offset such that the polarity of each field is counter to that of the adjacent field and each pair of fields is separated, at least in part, by an insulator and a thermal harnessing means for increasing kinetic energy in the active agent(s) or formulation including active agent(s).

5. A method of delivering an active agent(s) or a formulation including the active agent(s) to a patient comprising the step of:
(a) locating said active agent(s) or formulation including active agent(s) between a device and the subject, wherein the device comprises a biphasic array of magnetic materials wherein the magnetic fields generated by the magnetic materials are counter aligned such that the polarity of each field is counter that of the field adjacent to it and each pair of fields are separated by an insulator and a thermal harnessing means for increasing kinetic energy in the active agent(s) or formulation including active agent(s).

6. A method of claim 4 wherein the device is secured to the subject by a wearable and thermally insulating occlusive dressing or patch.

7. The device according to claim 2 wherein the device is secured to the subject by a wearable and thermally insulating occlusive dressing or patch.

8. The device according to claim 5 wherein the device is secured to the subject by a wearable and thermally insulating occlusive dressing or patch.
